(19) United States
(12) Patent Application Publication
(10) Pub. No.: US 2010/0076367 A1
(43) Pub. Date: Mar. 25, 2010

(54) IONTOPHORETIC DEVICE

(75) Inventors: Paul Anton Josef Ackermans, Nuenen (NL); Roelf Kassies, S-Hertogenbosch (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/515,541

(22) PCT Filed: Nov. 20, 2007
(86) PCT No.: PCT/IB2007/054710

§ 371 Date: May 20, 2009

(30) Foreign Application Priority Data
Nov. 24, 2006 (EP) 06124766.4

(51) Int. Cl. A61N 1/30

(52) U.S. Cl. 604/20; 132/200

(57) ABSTRACT

The invention relates to an iontophoretic device for the enhanced transport of substances through the skin (1), wherein at least two electrical transportation fields (Eₚ, Eₒ) with different directions are applied. In a preferred embodiment, these field are orthogonal and parallel to the skin surface, respectively, and applied in a temporally alternating sequence. Substances are thus transported in an optimal way on their meandering paths through the stratum corneum into or out of the skin. The iontophoretic device preferably comprises an array of electrodes (11) to which either a spatially alternating pattern of electrical potentials can be applied or which can alternatively be divided into two groups of neighboring electrodes that are connected to the same potential. The iontophoretic device may particularly be integrated into any skin-contacting device e.g. an electrical shaver.

The invention relates to an iontophoretic device and method for the enhanced transportation of substances through the skin. Moreover, it relates to a skin-contacting device comprising such an iontophoretic device.

Iontophoresis is a well known technology to enhance the transport of charged substances through the skin with the help of an electrical field. The U.S. Pat. No. 4,950,229 A discloses in this respect an iontophoretic device for the application of electrical fields that are perpendicular to the skin surface.

Based on this situation it was an object of the present invention to improve the efficiency of iontophoretic techniques.

This object is achieved by an iontophoretic device according to claim 1, a skin-contacting device according to claim 10, and a method according to claim 12. Preferred embodiments are disclosed in the dependent claims.

The iontophoretic device according to the present invention shall enhance the transport of certain substances through a region of the (human or animal) skin, wherein said transport may be directed from the outside into the body (e.g. when drugs or cosmetics shall be applied) or from the inside of the body to the outside (e.g. when analytes shall be sampled for diagnostic purposes). The substances to be transported are typically charged, for example ionic atoms or molecules with pharmaceutical or cosmetic function. In some cases uncharged particles may however be transported as well. The iontophoretic device comprises an electrode system and an associated controller for the generation of (at least) two electrical transportation fields in said region that are differently directed, wherein “different directions” of the electrical transportation fields are by definition assumed if the vectors of the electrical fields enclose at one point in space an angle between about 10° and 170°, preferably between about 30° and 150°. The electrode system usually comprises one or more single electrodes, for example metallic conductors that can be clamped to a certain electrical potential. The controller is typically connected by wires to the electrodes of the electrode system for supplying suitable electrical signals, e.g. voltages, to the electrodes. Moreover, it should be noted that the term “transportation field” serves just as a reference name indicating that this electrical field will usually have a function with respect to the desired transport of the substances through the skin.

While usual iontophoretic devices apply a static electrical field that has at a certain point within the skin always the same direction, the iontophoretic device described above operates with two electrical fields of different direction. This has the advantage that forces in corresponding different directions can be exerted on the substances to be transported, which helps to direct these substances along the irregular, tortuous paths they have to take particularly in the outermost layer (stratum corneum) of the skin.

According to a preferred embodiment of the iontophoretic device, the generated electrical transportation fields are orthogonal with respect to each other, wherein “orthogonality” is defined in a practical sense by an intersection angle of about 80° to 100°. Moreover, the iontophoretic device is preferably designed such that, when it is applied to a skin region, the generated electrical transportation fields are orthogonal and parallel to the skin surface, respectively. Such a design optimally takes into account the “brick and mortar” like structure of the stratum corneum, wherein the cells correspond to the bricks and wherein the substances have to follow routes along the mortar.

In general, the two differently directed electrical transportation fields may be present at the same time. In a further development of the invention, the controller of the iontophoretic device comprises however a mode-switching unit for selectively switching between different patterns of electrical potentials applied to the electrode system (or, more precisely, applied to the electrodes of the electrode system), wherein each of these patterns generates an electrical transportation field of the kind mentioned above. The differently directed electrical transportation fields will therefore not be generated simultaneously but in a temporal sequence. This guarantees that, at a certain point in time, only one of the fields acts on the substances to be transported. The patterns will in typically embodiments consist of only two different electrical potentials (“positive” and “negative”). They may however also comprise more than two different potentials (a different potential might even be applied to each electrode of the electrode system).

Thus the application of an electrical transportation field that is parallel to the skin surface can for example alternate with the application of an electrical transportation field that is orthogonal to the skin surface; the parallel field then enhances a transport along the different cell layers of the stratum corneum, while the orthogonal fields assist the substances to pass from one cell layer to another (one that is deeper in the skin if the transport is directed inwards).

The number and orientation of the applied electrical transportation fields as a well as the temporal pattern of their activation are typically optimized by theoretical considerations and/or experiments to achieve a maximal transport enhancement. Thus it is for example possible to apply two electrical transportation fields that are parallel and orthogonal to the skin surface, respectively, wherein the duration of the parallel field is preferably longer than the duration of the orthogonal field (e.g. in a ratio between 70:30 and 99:1). Such ratios take into account that substances have to travel comparatively long distances parallel and much shorter distances orthogonal to the skin surface (when crossing from one cell layer to the next). Moreover, the switching sequence executed by the mode-switching unit is preferably adapted or optimized with respect to a particular substance to be transported. Such an adaptation takes into account that different substances have different mobility in the applied electrical transportation field, which allows to optimally match the distances a substance is transported by a certain transportation field to the particular structure of the skin that has to be crossed (i.e. to the size and arrangement of the cells and the interstitial spaces).

According to a preferred embodiment of the invention, the electrode system comprises a plurality of single electrodes that are arranged in a one- or two-dimensional spatial array, wherein said arrangement is to be placed into contact with the skin surfaces above the region through which the substances have to be transported. Voltages between the different electrodes can then be used to generate electrical fields that have a desired course in the region of interest.

In a combination of the embodiments of an iontophoretic device with a mode-switching unit and with an electrode array, one pattern of electrical potentials preferably comprises the application of at least two different potentials (“positive” and “negative”) in a spatially alternating way to the array of electrodes. Thus a kind of one- or two-dimensional chessboard pattern consisting of positive and negative potentials can be generated, which induces electrical fields in the skin below the electrodes that are mainly parallel to the skin surface. It should be noted that the potentials need not necessarily change between every pair of neighboring electrodes, i.e. there may be groups of neighboring electrodes having the same potential. The overall pattern should however have an alternating character, which is for example achieved if the mentioned groups of neighboring electrodes with the same potential do not comprise more than 10% of the total number of electrodes.

In another embodiment of the iontophoretic device with a mode-switching unit and an electrode array, one pattern of potentials comprises the application of different potentials to groups of spatially neighboring electrodes, wherein said groups of neighboring electrodes with the same potential typically each comprise more than 10% of the total number of electrodes. If there are only two such groups, the whole array of single electrodes is effectively divided into a design with two large “meta-electrodes” having different potentials. In the skin region under these meta-electrodes, the electrical field is primarily oriented orthogonal to the skin surface.

The iontophoretic device may optionally comprise a reservoir for substances to be transported into the skin and/or to be sampled from the skin.

The invention further relates to a skin-contacting device, i.e. a device that is at least partially brought into contact with the skin during its application. Typical examples of such a skin-contacting device are an applicator for pharmaceuticals or cosmetics, an electrical shaver, a manual shaver, an epilator, a cosmetic patch and an applicator for a sunscreen. The device comprises an iontophoretic device of the kind described above, i.e. an iontophoretic device comprising an electrode system and an associated controller for the generation of two electrical transportation fields that are differently directed, preferably mutually orthogonal. With such a skin-contacting device, a medical or cosmetic skin-care substance can be applied to the skin or some biological substance can be sampled from the body in a very efficient way. If the device has some additional function, e.g. in case of a shaver or epilator, this additional function and the iontophoretic application can be achieved simultaneously without an extra procedure. It is however of crucial importance in this case that the desired iontophoretic transport of substances can be achieved during the available short time, e.g. the time that is needed for shaving a certain area of the skin. This aim can favorably be met by the application of differently directed electrical transportation fields as it was described above.

Preferred embodiments of the skin-contacting device are analogous to the preferred embodiments of the iontophoretic device described above and will therefore not be repeated in detail.

The skin-contacting device will usually have some handgrip by which a user can hold it during its use. Optionally at least one electrode of the electrode system may be located in this handgrip, thus closing the electrical circuit via the body of the user. In case the skin-contacting device is an electrical or manual shaver, the electrodes of the electrode system may be arranged on it in many different ways. According to one preferred embodiment, at least one electrode of the electrode system is located in the shaving head of the shaver or in the mounting of this shaving head. In case of an electrical shaver, the “shaving head” consists by definition of the parts that are moved over the skin during shaving; it comprises of components (particularly the shaving caps with lamellae) that are more or less fixed to the device and that move over the skin due to the shaving movements made by the user. Cutting blades move behind the lamellae, driven by the electrical motor of the shaver.

The invention further relates to a method for the iontophoretic enhancement of the transport of substances through a region of the skin, wherein said method comprises the application of differently directed electrical transportation fields to said region. The method comprises in general form the steps that can be executed with a iontophoretic device of the kind described above. Therefore, reference is made to the preceding description for more information on the details, advantages and improvements of that method.

In a preferred embodiment of the method, the electrical transportation fields are applied in a temporally alternating sequence such that the substances to be transported are forced in one definite direction at each point in time.

In a further development of the aforementioned approach, the alternating sequence of applied electrical transportation field is adapted to the substance to be transported, to the electrode geometry and/or to a particular skin structure. Thus the individual mobility of substances can for example be exploited to realize a substance-specific transport enhancement.

These and other aspects of the invention will be apparent from and elucidated with reference to the embodiment(s) described hereinafter. These embodiments will be described by way of example with the help of the accompanying drawings in which:

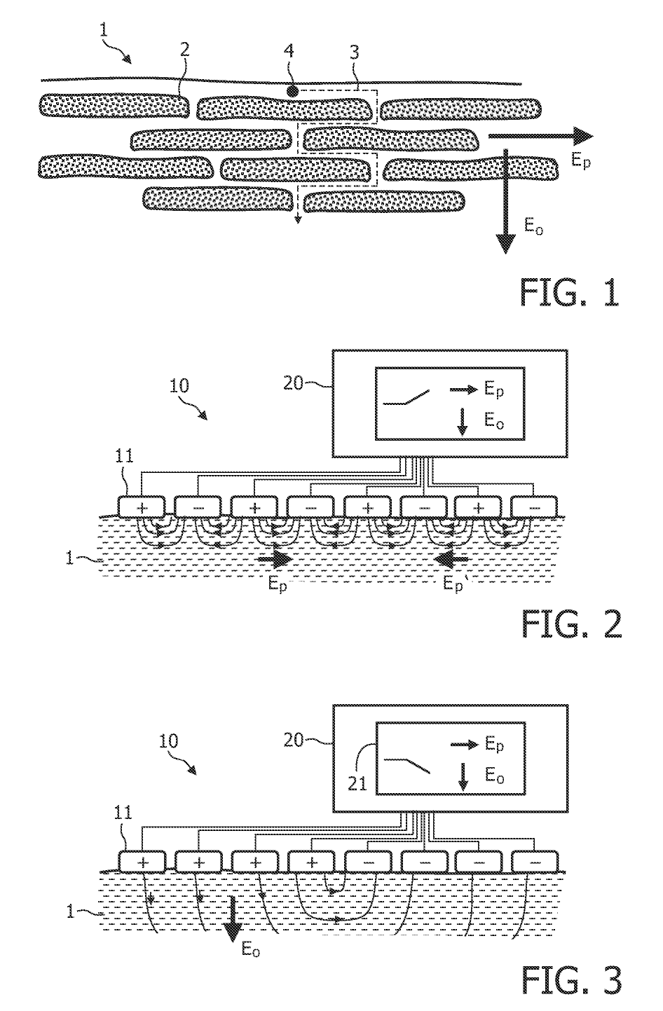

FIG. 1 shows a schematic cross section through the stratum corneum;

FIG. 2 shows an iontophoretic device according to the present invention that is applied in an operating mode with electrical transportation fields parallel to the skin surface;

FIG. 3 shows the iontophoretic device of FIG. 2 that is applied in an operating mode with electrical transportation fields orthogonal to the skin surface;

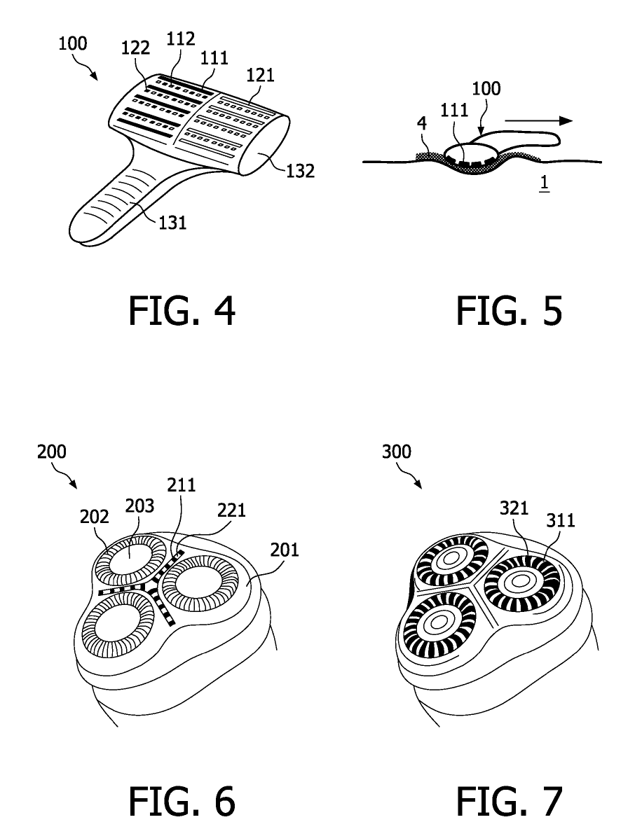

FIG. 4 shows a schematic perspective of a general applicator for cosmetic or pharmaceutical substances;

FIG. 5 shows a section through the general applicator of FIG. 4 during its operation;

FIG. 6 shows a first embodiment of an electrical shaver comprising an iontophoretic device with electrodes between the shaving heads;

FIG. 7 shows a second embodiment of an electrical shaver comprising an alternating arrangement of electrodes in the shaving head;

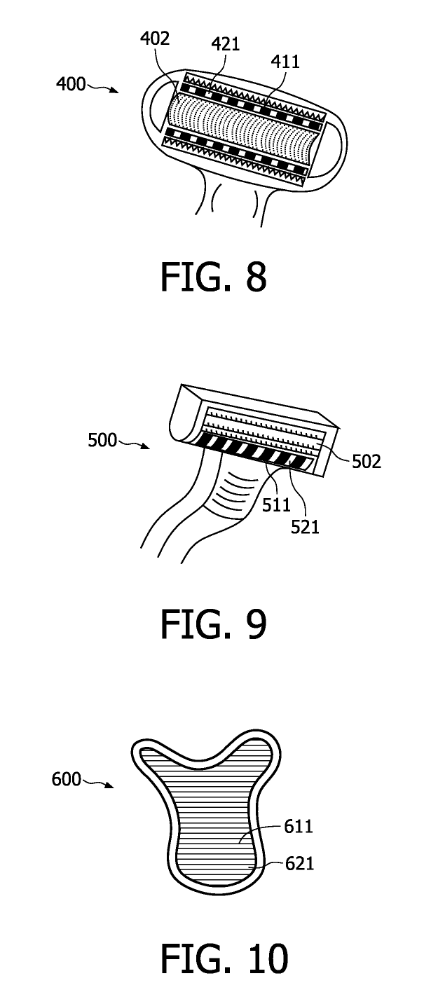

FIG. 8 shows a lady shaver with an iontophoretic device;

FIG. 9 shows a manual shaver with an iontophoretic device;

FIG. 10 shows a cosmetic patch with an iontophoretic device.

Like reference numbers or numbers differing by integer multiples of 100 refer in the Figures to identical or similar components.

Iontophoresis is a technology to enhance transport of charged substances through the skin. The following description will in this respect particularly consider the transport of substances (e.g. pharmaceuticals or cosmetics) from the outside of the body to the inside, though the inverse transport direction may be enhanced as well, e.g. if biological substances shall be sampled from the tissue for diagnostic purposes. FIG. 1 shows a schematic section through the outermost layer 1, the stratum corneum, of the skin. The stratum corneum consists of flat cells (the corneocytes 2), held together by lipid layers, comparable to a brick and mortar structure. The substances 4 to be delivered to the skin have to follow tortuous routes 3 along the “mortar”, called the transcellular pathway.

In general, the challenge in iontophoresis for substance delivery is to enhance the flux of a substance into the skin as much as possible, while at the same time keeping the induced skin irritation as low as possible. A related challenge in iontophoresis for substance delivery is connected to the formulation of the substance below the electrodes. In principle, all charged particles in the formulation are forced through the skin, not only the ones that are the effective ones, i.e. there is a competition between “wanted” and “unwanted” ions.

Due to the size of the electrodes, the electrical field lines Eₒ in traditional iontophoresis are orthogonal to the skin surface. This means that the particles are only accelerated in the direction perpendicular to the skin surface. However, there is a much larger part of the pathway that is parallel to the skin surface. In this part transport of the substances depends on (slow) passive diffusion only which thus limits the rate of transport. Moreover, it would be advantageous to have a sort of sieve to select only the “wanted” ions from an iontophoretic formulation.

To address the aforementioned items, it is proposed that an additional electrical field Eₚ parallel to the skin surface is added to the iontophoretic system. This parallel electrical field Eₚ will enhance the transport of ions in the part of the pathway parallel to the skin surface (which is substantially longer than the perpendicular part). It can be switched on and off alternating to the orthogonal field Eₒ. Calculations show that adding such a parallel electrical field Eₚ dramatically increases the substance flow through the skin and in addition reduces the time it takes to pass the stratum corneum.

In addition, the switching rate between the orthogonal and the parallel fields, Eₒ and Eₚ, can be chosen accurately with respect to the mobility of the wanted ions, allowing an enrichment of the wanted ions with respect to the unwanted ones. The frequency of switching should in this case be chosen such that the wanted ions get precisely enough time to travel horizontally to the next perpendicular part of their tortuous path. When then the orthogonal field Eₒ is switched on, the ions are ready to proceed perpendicularly, without being hampered by the corneocytes.

FIGS. 2 and 3 show particular realizations of an iontophoretic device according to the above principles. This device comprises an electrode array or system 10 placed on top of the skin 1 and an associated controller 20. The parallel electrical field Eₚ can be made as shown in FIG. 2 if the series of small electrodes 11 next to each other is put at opposite voltages (“+” and “−” signs).

However, if the voltages on the electrodes 11 are applied in two spatially connected groups (one with positive voltage and the other with negative voltage), as shown in FIG. 3, an orthogonal field E₀ can be created. The sequence of parallel and orthogonal fields is controlled by a mode-switching unit 21 of the controller 20.

The shown arrangement of electrodes and the pattern of potentials is of course only exemplary and can be modified in many ways and optimized towards actual applications (e.g. wider spaces between electrodes, a third electrode for the initial skin penetration, application of more than two different potentials, patterns of potentials p and n like pnnpnnp . . . , ppnppnpp . . . , or ppnnppnnpp . . . , etc.).

The invention can be used in all applications that are suitable for iontophoresis. These range from the cosmetic area for the enhanced delivery of cosmeceuticals to the medical field with applications e.g. in pain management and delivery of drugs for Parkinson's disease. As was already mentioned, the described approach may also be applied to the enhanced sampling of substances from the body, which offers various advantages: (a) the lag time will be shorter than in traditional iontophoresis, (b) the rate of transport can be higher and (c) selective transport of a chosen analytes can be achieved by tuning the switching frequency between horizontal and vertical fields properly.

FIGS. 4 to 10 illustrate some particular applications of the described iontophoretic principle, wherein the shown examples are far from complete.

FIGS. 4 and 5 show a general applicator 100 for applying pharmaceutical or cosmetic substances 4 to the skin 1. It comprises a hand grip 131 an a head 132 that carries an arrangement of electrodes to which suitable patterns of potentials (indicated by black and white in the Figure) can be applied. The head may in particular comprise two blocks with
linear electrodes 111, 121 arranged in parallel one below the other, wherein different potentials can e.g. be applied to the electrodes of the first and the second block, respectively;
dot-shaped electrodes 112, 122 arranged in line between the linear electrodes 111, 121, wherein different potentials can e.g. be applied to these electrodes in an alternating sequence.

In this embodiment, it is also possible to realize the alternation of orthogonal and parallel fields not only by switching electrically, but also as a result of the movement of the device over the skin. The different parts of the electrode geometry in the device pass each point in the skin sequentially.

As FIG. 5 shows, a substance 4 can efficiently be transported into the body by moving the applicator 100 over the skin.

FIG. 6 relates to an application where an iontophoretic device is integrated into an electrical shaver 200. The shaver 200 comprises a handgrip and a top, wherein the top consists of (i) three annular shaving caps 202 which contain rotating blades behind stationary lamellae, (ii) an outer mounting 201 around the shaving caps 202, and (iii) an inner circular mounting 203 within the shaving caps which is called “decocap”. In FIG. 6, a star-shaped segmented electrode system is disposed between the shaving heads 202. This segmented electrode system comprises both poles 211 and 212 of the iontophoretic device. The shaving cream or additive to be transported through the skin are applied to the skin before or during shaving. While moving the shaver head through the additive over the skin during the shaving process, the active ingredients are delivered in the skin by means of the electric current.

FIG. 7 shows a modification of the embodiment of FIG. 6, where the anodes and cathodes are implemented on adjacent lamella 311, 321 of the shaving heads of an electrical shaver 300. This compact electrode geometry generates a very superficial electrical field inside the skin.

In the lady shaver 400 shown in FIG. 8, two electrode array strips of an iontophoretic device are located on either side of the cutting element 402. The electrode arrays hold again both poles 411 and 421.

FIG. 9 shows a blade shaver 500 as an example of a manual shaver, in which the opposite electrodes 511 and 521 of an integrated iontophoretic device are arranged in an array geometry below the blades. Manual shavers are available in many different styles, for example disposable, disposable cartridge, straight razor, blade razor and safety razor (using single- or double-edged blades).

FIG. 10 illustrates a cosmetic patch 600 with electrodes 611 and 621 as a further example of a skin-contacting product with an integrated iontophoretic device.

For all embodiments, the time profile of the electrical current has to be tuned to the combination of electrode geometry, skin structure and substance mobility to obtain optimal delivery.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1. A iontophoretic device for the enhanced transport of substances through a region of the skin (1), comprising an electrode system (10) and an associated controller (20) for the generation of two electrical transportation fields (Eₚ, Eₒ) in said region that are differently directed,
wherein the controller (20) comprises a mode-switching unit (21) for selectively switching between different patterns of electrical potentials applied to the electrode system (10), wherein each of the patterns generates one of the electrical transportation fields (Eₚ, Eₒ), wherein the electrical transportation fields (Eₚ, Eₒ) are applied in a temporally alternating sequence.

2. The iontophoretic device according to claim 1,
wherein the electrical transportation fields (Eₚ, Eₒ) are mutually orthogonal.

3. The iontophoretic device according to claim 1,
wherein the electrical transportation fields (Eₚ, Eₒ) are parallel and orthogonal to the skin (1) surface, respectively, when the device is applied.

4. (canceled)

5. The iontophoretic device according to claim 1,
wherein the switching sequence executed by the mode-switching unit (21) is adapted to a particular substance, electrode geometry and/or skin structure.

6. The iontophoretic device according to claim 1,
wherein the electrode system (10) comprises a plurality of single electrodes (11) arranged in a spatial array.

7. The iontophoretic device according to claim 5,
wherein one pattern comprises the application of at least two different potentials in a spatially alternating way to the array of electrodes (11).

8. The iontophoretic device according to claim 5,
wherein one pattern comprises the application of different potentials to groups of spatially neighboring electrodes (11).

9. The iontophoretic device according to claim 1,
wherein it comprises a reservoir for substances to be transported into the skin and/or sampled from the skin.

10. A skin-contacting device, comprising an iontophoretic device as claimed in claim 1, wherein the device comprises one of:
an applicator for pharmaceuticals or cosmetics (100;
an electrical shaver (200, 300, 400);
a manual shaver (500);
an epilator;
a cosmetic patch (600); or
a sunscreen applicator.

11. The skin-contacting device according to claim 10, particularly in the form of an electrical or manual shaver (200, 300, 400, 500),
wherein at least one electrode of the electrode system is located in the handgrip of the device and/or in a shaving head of the shaver or its mounting.

12. A method for the iontophoretic enhancement of the transport of cosmetic substances through a region of the skin (1), comprising the application of differently directed electrical transportation fields (Eₚ, Eₒ) to said region using an electrode system, wherein the method comprises selectively switching between different patterns of electrical potentials applied to the electrode system (10), wherein each of the patterns generates one of the electrical transportation fields (Eₚ, Eₒ), wherein the electrical transportation fields (Eₚ, Eₒ) are applied in a temporally alternating sequence.

13. (canceled)

14. The method according to claim 11,
wherein the alternating sequence is adapted to the substance to be transported, electrode geometry and/or skin structure.
